(19) United States
(12) Patent Application Publication
(10) Pub. No.: US 2026/0315009 A1
(43) Pub. Date: Oct. 15, 2026

(54) SYSTEMS AND METHODS FOR AN AUTOREGRESSIVE SCHEME FOR SYNTHETIC TIME-SERIES GENERATION UNDER DATA SCARCITY

(71) Applicant: ARIZONA BOARD OF REGENTS ON BEHALF OF ARIZONA STATE UNIVERSITY, Tempe, AZ (US)

(72) Inventors: Hassan Zadeh, Phoenix, AZ (US); Asiful Arefeen, Phoenix, AZ (US)

(21) Appl. No.: 19/491,528

(22) PCT Filed: Jun. 14, 2024
(86) PCT No.: PCT/US2024/034077

§ 371 Date: Dec. 9, 2025

Related U.S. Application Data
(60) Provisional application No. 63/508,240, filed on Jun. 14, 2023.

(51) Int. Cl. A61B 5/00; G06N 3/08; G16H 20/17

(52) U.S. Cl. CPC A61B 5/7278; G06N 3/08; G16H 20/17

(57) ABSTRACT

An autoregressive framework for synthetic data generation using an autoregressive generative model as an alternative to Generative Adversarial Networks (GAN). The autoregressive framework is applied to continuous glucose monitor data uses small real datasets to generate synthetic glycemic responses. The framework performs better than other systems because it works well with data constrained environments and can also evaluate contextual values in addition to past values.

### CROSS-REFERENCE TO RELATED APPLICATIONS

This is a U.S. Non-Provisional Patent Application that claims benefit to U.S. Provisional Patent Application Ser. No. 63/508,240 filed 14 Jun. 2023, which is herein incorporated by reference in its entirety.

### GOVERNMENT SUPPORT

This invention was made with government support under 2210133 awarded by the National Science Foundation. The government has certain rights in the invention.

### FIELD

The present disclosure generally relates to synthetic data generation, and in particular, to a system and associated method for synthetic time-series generation under data scarcity using an autoregression scheme.

### BACKGROUND

Medical data scarcity continues to be a problem for biomedical data scientists and the healthcare industry because many innovative biomedical solutions rely on high-quality medical data for health monitoring, health assessment, and clinical decision making. The limited availability of high-quality medical data is a hindrance for the development and deployment of innovative solutions to some of the most pressing healthcare problems. Shortage of medical data largely stems from different privacy constraints, high cost of data collection, prolonged annotation periods, problems with data quality, and sharing difficulties. The full potential of medical data-driven research, diagnosis, and treatment can be unlocked by tackling most of the aforementioned issues.

An example of medical data scarcity is the limited availability of clinical studies. Clinical studies are essential for the development and approval of new medical technologies, treatments and devices. However, they must undergo extensive institutional review board (IRB) approval processes, take a long time to complete and, are conducted with a limited number of participants who are not representative of the general population. Additionally, only a fraction of clinical trials data is publicly available, and access to the rest is often restricted by proprietary interests.

Another illustration can be given from the standpoint of chronic diseases. Using continuous glucose monitor (CGM) data has been shown to be valuable for effective diabetes management as they transmit blood glucose data at a regular interval. CGM datasets also include nutrition labels, however, their annotation imposes more hardship than just collecting it because of the need for domain experts and processing of the raw data noted down by the subjects. While several unlabeled CGM datasets are available, a handful of labeled CGM datasets are accessible to the researchers despite their significance. Thanks to numerous challenges including IRB permission, limited patients, effort of annotation and under representative samples of the general population, obtaining a CGM dataset through clinical studies of patients with diabetes appears to be difficult. Regardless of these shortcomings, study and development in this field are valuable because of the potential advantages of using CGM data to control diabetes.

It is with these observations in mind, among others, that various aspects of the present disclosure were conceived and developed.

### SUMMARY

A method for generating synthetic time-series physiological signal data includes: accessing a set of context data; and iteratively sampling, by an autoregressive model and for a t-th time-step of a plurality of time-steps, a t-th value of a synthetic time-series signal from a conditional probability distribution. Sampling the t-th value from the conditional probability distribution can be based on (e.g., in view of or otherwise conditioned on): the set of context data; a set of distribution parameters of the autoregressive model that characterize the conditional probability distribution; and one or more previous values of the synthetic time-series signal for one or more previous time-steps of the plurality of time-steps. In some examples, the synthetic time-series signal is a representation of a synthetic continuous glucose monitoring (CGM) signal.

The method can further include: applying the synthetic time-series signal as training data for a neural network model implementing a downstream task.

For training the autoregressive model, the method can include: accessing a training dataset that includes a training time-series signal having a plurality of training time-steps; accessing a set of training context data that correlates with the training time-series signal; and optimizing, over a plurality of training iterations, the set of distribution parameters of the autoregressive model to generate a synthetic training time-series signal that correlates with a probability distribution of observing the training time-series signal in view of the set of training context data.

The optimization step for training the autoregressive model can include: determining, at a t-th training iteration of the plurality of training iterations and for a t-th training time-step of the plurality of training time-steps, a t-th set of distribution parameters of the set of distribution parameters of the autoregressive model, the t-th set of distribution parameters characterizing a t-th conditional probability distribution associated with observing a t-th value of the training time-series signal under the set of training context data and in view of one or more previous values of the training time-series signal for one or more previous training time-steps of the plurality of training time-steps; and sampling, at the t-th training iteration of the plurality of training iterations and for the t-th training time-step of the plurality of training time-steps, a t-th value of the synthetic training time-series signal from the t-th conditional probability distribution based on the set of training context data and the t-th set of distribution parameters.

Further, the optimization step for training the autoregressive model can include: optimizing a loss function that compares the synthetic training time-series signal with the training time-series signal and maximizes, for each training time-step of the plurality of training time-steps, a log-likelihood of observing a t-th value of the training time-series signal under a t-th set of distribution parameters.

For generating new context data that can be applied as input to the autoregressive model (e.g., in order to generate the synthetic time-series signal that correlates with the new context data, the method can further include: accessing a set of existing context data; and constructing a set of new context data based on the set of existing context data. The set of context data for sampling the t-th value of the synthetic time-series signal can include the set of new context data.

The step of constructing a set of new context data can include various sub-steps, including: extracting a first group of context data for inclusion in the set of new context data from the set of existing context data; generating a second group of context data for inclusion in the set of new context data from the set of existing context data as random values sampled from an acceptable range in view of the first group of context data; and generating a third group of context data for inclusion in the set of new context data by application of one or more instances from the first group of context data and/or the second group of context data as input to a machine learning model. The machine learning model can include a polynomial regression model and/or a kNN regression model.

In some examples, the third group of context data includes insulin dose amount generated in view of the first group of context data including food items and corresponding macronutrients associated with the food items. The third group of context data can also include time since last insulin dose generated in view of: the first group of context data including food items and corresponding macronutrients associated with the food items; the second group of context data including pre-meal blood glucose levels; and/or the third group of context data including insulin dose amount.

In a further aspect, a system for generating synthetic time-series physiological signal data includes: a processor in communication with a memory, the memory including instructions executable by the processor to implement aspects of the method above. These include instructions executable by the processor to: access a set of context data; and iteratively sample, by an autoregressive model and for a t-th time-step of a plurality of time-steps, a t-th value of a synthetic time-series signal from a conditional probability distribution. Sampling the t-th value from the conditional probability distribution can be based on (e.g., in view of or otherwise conditioned on): the set of context data; a set of distribution parameters of the autoregressive model that characterize the conditional probability distribution; and one or more previous values of the synthetic time-series signal for one or more previous time-steps of the plurality of time-steps. In some examples, the synthetic time-series signal is a representation of a synthetic continuous glucose monitoring (CGM) signal.

Further, the memory can include instructions executable by the processor to: apply the synthetic time-series signal as training data for a neural network model implementing a downstream task.

For training the autoregressive model, the memory can include instructions executable by the processor to: access a training dataset that includes a training time-series signal having a plurality of training time-steps; access a set of training context data that correlates with the training time-series signal; and optimize, over a plurality of training iterations, the set of distribution parameters of the autoregressive model to generate a synthetic training time-series signal that correlates with a probability distribution of observing the training time-series signal in view of the set of training context data.

The memory can further include instructions executable by the processor to: determine, at a t-th training iteration of the plurality of training iterations and for a t-th training time-step of the plurality of training time-steps, a t-th set of distribution parameters of the set of distribution parameters of the autoregressive model, the t-th set of distribution parameters characterizing a t-th conditional probability distribution associated with observing a t-th value of the training time-series signal under the set of training context data and in view of one or more previous values of the training time-series signal for one or more previous training time-steps of the plurality of training time-steps; and sample at the t-th training iteration of the plurality of training iterations and for the t-th training time-step of the plurality of training time-steps, a t-th value of the synthetic training time-series signal from the t-th conditional probability distribution based on the set of training context data and the t-th set of distribution parameters.

The memory can further include instructions executable by the processor to: optimize a loss function that compares the synthetic training time-series signal with the training time-series signal and maximizes, for each training time-step of the plurality of training time-steps, a log-likelihood of observing a t-th value of the training time-series signal under a t-th set of distribution parameters.

For generating new context data that can be applied as input to the autoregressive model (e.g., in order to generate the synthetic time-series signal that correlates with the new context data, the memory can further include instructions executable by the processor to: access a set of existing context data; and construct a set of new context data based on the set of existing context data. The set of context data for sampling the t-th value of the synthetic time-series signal can include the set of new context data.

To construct the set of new context data the memory can further include instructions executable by the processor to: extract a first group of context data for inclusion in the set of new context data from the set of existing context data; generate a second group of context data for inclusion in the set of new context data from the set of existing context data as random values sampled from an acceptable range in view of the first group of context data; and generate a third group of context data for inclusion in the set of new context data by application of one or more instances from the first group of context data and/or the second group of context data as input to a machine learning model. The machine learning model can include a polynomial regression model and/or a kNN regression model.

In some examples, the third group of context data includes insulin dose amount generated in view of the first group of context data including food items and corresponding macronutrients associated with the food items. The third group of context data can also include time since last insulin dose generated in view of: the first group of context data including food items and corresponding macronutrients associated with the food items; the second group of context data including pre-meal blood glucose levels; and/or the third group of context data including insulin dose amount.

In a further aspect, a non-transitory computer readable media includes instructions encoded thereon that are executable by the processor to perform aspects of the methods outlined herein, including: accessing a set of context data; and iteratively sampling, by an autoregressive model and for a t-th time-step of a plurality of time-steps, a t-th value of a synthetic time-series signal from a conditional probability distribution. Sampling the t-th value from the conditional probability distribution can be based on (e.g., in view of or otherwise conditioned on): the set of context data; a set of distribution parameters of the autoregressive model that characterize the conditional probability distribution; and one or more previous values of the synthetic time-series signal for one or more previous time-steps of the plurality of time-steps. In some examples, the synthetic time-series signal is a representation of a synthetic continuous glucose monitoring (CGM) signal.

### BRIEF DESCRIPTION OF THE DRAWINGS

FIG. 1 is a graphical representation showing a physiological signal and its corresponding context (meal, medication and health information);

FIGS. 2A and 2B are a pair of diagrams showing a system for implementation of an autoregressive scheme for synthetic time-series generation under data scarcity;

FIG. 3A is a simplified diagram showing training and data synthesis of a synthetic time-series generation component of the system of FIGS. 2A and 2B;

FIG. 3B is a diagram showing a training process of the synthetic time-series generation component of FIG. 3A;

FIG. 3C is a simplified diagram showing an architecture of one embodiment of an autoregressive model of the synthetic time-series generation component of FIG. 3A;

FIG. 4 is a simplified diagram showing a context generation component of the system of FIGS. 2A and 2B including three subsets (groups) of context data;

FIG. 5 is a graphical representation demonstrating no significant correlation is found between the carbohydrate amount and pre-meal blood glucose level;

FIG. 6 is a graphical representation demonstrating that strong correlation exists between the carbohydrate amount and pre-meal insulin intake;

FIG. 7 is a graphical representation showing a visualization of a synthetic signal produced by slightly perturbing the context of a real CGM used to train the model, where the synthesized signal replicates the pattern of the parent signal;

FIGS. 8A-8E are a series of graphical representations showing comparison of mean value distribution between real and synthetic CGM data for 5 subjects using KDE plots, where the synthetic means generated by the autoregressive model closely resemble those of the real data;

FIG. 9 is a graphical representation showing an area under the CGM curve increasing as consumed CHO amount increases with other parameters fixed, data taken from subject P2 (see FIG. 8B);

FIG. 10 is a graphical representation showing an area under the CGM curve decreasing as insulin intake increases with other parameters fixed, data taken from subject P1 (see FIG. 8A);

FIG. 11 is a process flow diagram showing a method for generating synthetic time-series physiological signal data by the system of FIGS. 2A-4;

FIG. 12 is a process flow diagram showing a sub-method of the method of FIG. 11 for training the autoregressive model of FIGS. 3A-3C;

FIG. 13 is a process flow diagram showing a sub-method of the method of FIG. 11 for generating context data by the context generation component of FIG. 4; and

FIG. 14 is a simplified diagram showing an example computing device for implementation of the system of FIGS. 2A and 2B.

Corresponding reference characters indicate corresponding elements among the view of the drawings. The headings used in the figures do not limit the scope of the claims.

### DETAILED DESCRIPTION

Medical data acquisition is challenging due to privacy concerns, data collection costs, data quality issues, and data sharing complications. Additionally, availability of biomedical data that combines physiological measure-ments with individual's contextual factors is limited.

To overcome these challenges, the use of synthetic data that closely resembles real data has long been a desirable approach in data-driven modeling. While Generative Adversarial Networks (GANs) and Diffusion models are known as cutting edge techniques for producing synthetic data, they struggle to work with very small datasets. Thus, there is a need for models, which suit small datasets better.

The present disclosure outlines GlySynth, an autoregressive framework to synthesize time-series physiological data from contextual information. An advantage of autoregressive models is their ability to perform well on small datasets. In one example implementation, the autoregressive framework was trained using a small continuous glucose monitoring (CGM) dataset of only 167 samples to map artificial CGM signals belonging to new context, i.e., specific meals, medications and health status. Furthermore, this disclosure outlines how new contextual information can be effectively developed to feed the model for synthesizing new time-series via inference. Results demonstrate the effectiveness of the autoregressive approach through statistical comparisons with real data, causality test, as well as its ability to accurately estimate meal macro-nutrient contents. Inclusion of synthetic data significantly enhances the Root Mean Square Relative Error (RMSRE) of carbohydrate estimation, achieving an impressive improvement up to 0.22.

### 1. Introduction

The scarcity of high-quality, diverse, and accessible data is a major bottleneck that affects the ability of algorithms and models to learn from and generalize to new data. Lack of data can result in overfitting, where the model fits the training samples too closely rather than learning the underlying patterns in the data. Therefore, the model is unlikely to generalize well to new, unseen data. Model's performance is also likely to be impeded by high variance, meaning it will be sensitive to small changes in the training data. This can again result in significant variations in the model's effectiveness on different samples. Furthermore, the model is likely to have low bias, meaning it is unlikely to make consistent predictions across different samples from same distribution.

Therefore, there is an unmet need to obtain data for training a neural network. This is where synthetic data comes into play. Synthetic data is defined as artificially generated data that closely resembles real data and can be used to address the data scarcity challenges. Studies show that using synthetic data can significantly assist data scientists overcome the drawbacks of using small datasets by introducing diversity and increasing the robustness, generalization and adaptability of data-driven AI models. If quality data is available in limited quantity, there is a need to replicate the data synthetically. Furthermore, synthetic data can potentially reduce the risk of privacy breaches since it does not contain sensitive information.

However, considerations must be taken into account to ensure that the data generation process results in high quality data since low-quality synthetic data might lead to biased models. For example, if the training data used to generate synthetic data is not representative of the true population and does not take into account domain knowledge (e.g., range of carb), the resulting synthetic data may not accurately reflect the underlying distribution of the data. This emphasizes the significance of creating high-quality synthetic data using advanced methods like GANs and Diffusion models. Nevertheless, thanks to their high levels of instability and difficulty in training, GANs and Diffusion models perform poorly when working with relatively tiny datasets. In contrast, the present disclosure outlines autoregressive models which work well under data constrained environment. Autoregressive models are based on the principle of using past observations to predict future values, meaning that each projected value in an autoregressive model is the weighted sum of the previous several values in the series. When there is a clear temporal structure to the data and the future value of a time-series draws information from previous values, autoregressive models are very helpful. Autoregressive models can also be extended to draw additional information from contextual values instead of just using the past values.

Contributions can be summarized as follows:

An autoregressive framework for synthetic data generation to serve as a direct competitor to GANs. The framework boasts the ability to perform well in data constrained environments.

A tiny dataset can be leveraged to train an autoregressive model. Further, machine learning based regression approaches are outlined to generate new contexts and feed them to the trained autoregressive model for data synthesis.

Several machine learning-based regression approaches are also provided to process the contextual data and feed them for data synthesis. Performance of the framework is demonstrated via statistical comparisons of synthetic data to real data and by verifying if the framework grasps the causal relation between the inputs and outputs. Additionally, the quality of the synthetic data is evaluated through its use in a downstream regression task of macro-nutrient estimation.

### 2. Problem Statement

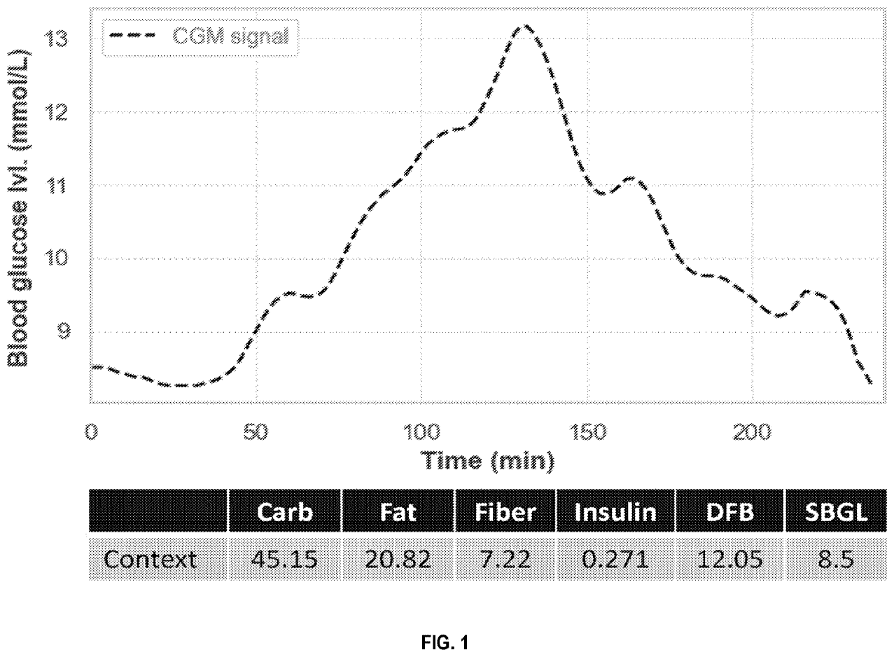

Synthetic data generation remains a challenge in data science. Traditional methods for generating synthetic data often lead to inconsistent and unrealistic data in data-limited setup, affecting the performance of downstream machine learning algorithms negatively. Assume that there is a small set of features/contextual information and their corresponding time-series signals recorded with sensors while the subjects were in a clinical trial (FIG. 1). The contexts can be represented in a feature space and they can be used in training machine learning algorithm along with the sequences. As they are very small in number, the likelihood is very high that the trained algorithm will perform poorly generalizing on new data. Although synthesizing new data may serve as a solution, data synthesis with very few instances remains another challenge. Recent research advocates the idea of training GANs with fewer samples, yet, at least 2k samples are often needed to produce meaningful and quality data using GANs. Therefore, quality data synthesis in a constrained environment is a challenge that must be addressed to enable computational modeling on time-series health data. This problem can be phrased as follows

Problem I (Constrained data synthesis): Let Y be sensor observations for t timestamps, represented as a time-series sequence i.e. Y={y₁, y₂, . . . , yₜ}. Furthermore, let X be a set of n features that provide contextual information for Y. Hence, X={X₁, X₂, . . . , Xₙ}. The goal is to use a generative model to produce realistic synthetic time-series sequences Z={z₁, z₂, . . . , zₜ}. Given a few samples constrained by a trivial number D, the model must be able to accurately capture the relationship between X, Y, and Z, such that the generated time-series Z is a probability distribution over sequences given X and Y. The main challenge in solving this constrained data synthesis problem is to identify, of all the possible generative models, what model to use under extreme data scarcity.

Problem II (Realistic contextual values): Even if there was a model for data synthesis with low data requirement, this model needs to be fed with new context information to ensure mass data generation. For example, producing realistic time-series sequences Z={z₁, z₂, . . . , zₜ} would require new context X*=

$$\{ {X}_{1}^{*} , {X}_{2}^{*} , … , {X}_{n}^{*} \}$$

for the model. For quality data generation, these contextual values need to be realistic as well. Now, question remains, how to get the new context to guarantee rapid and quality data production?

### 3. Solution and System Overview

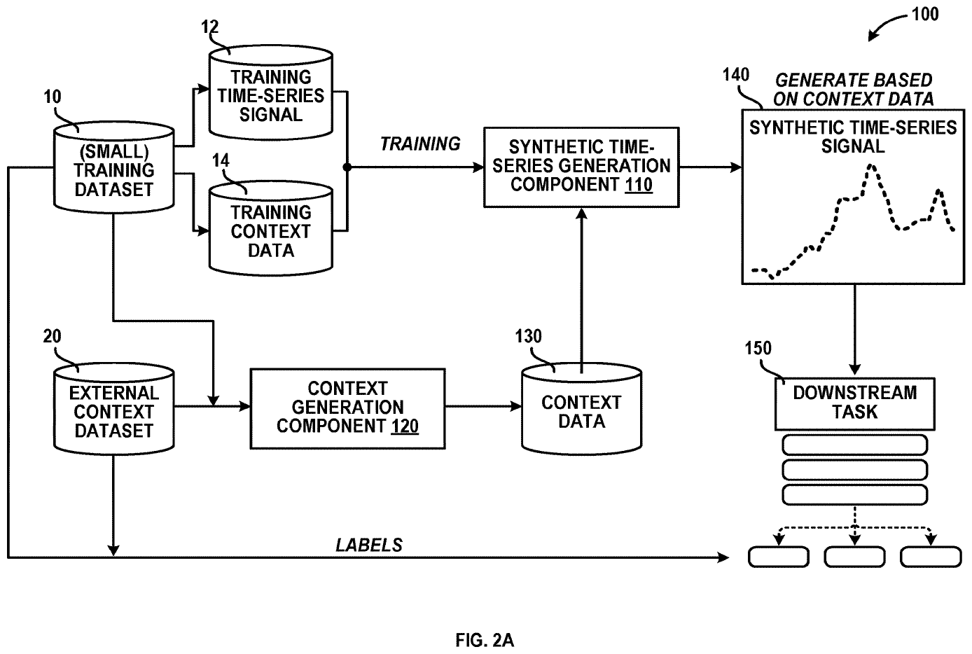

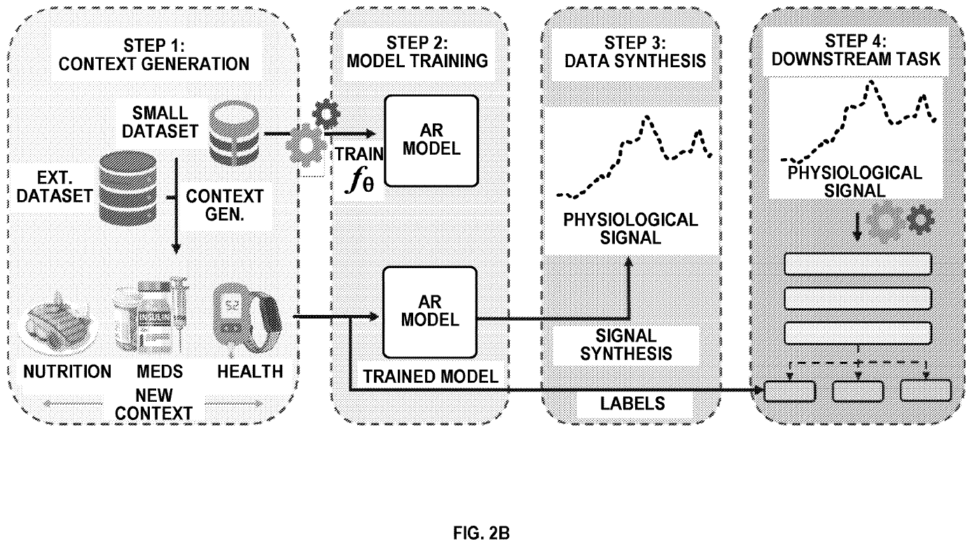

FIGS. 2A and 2B show a framework for generating synthetic physiological signals. The framework includes a generative model (autoregressive model) trained using with the small dataset that is needed to be amplified. As depicted in FIG. 2A, the trained model relies on synthetic context data (“new context”) to generate the synthetic physiological signals. These new contexts can be drawn either from an external dataset or through applying machine learning techniques that extract relevant information from the external data. With the new contexts available, new physiological data can be synthesized at scale. The final step involves validating the quality of the synthetic data in a downstream task, ensuring their usefulness and reliability.

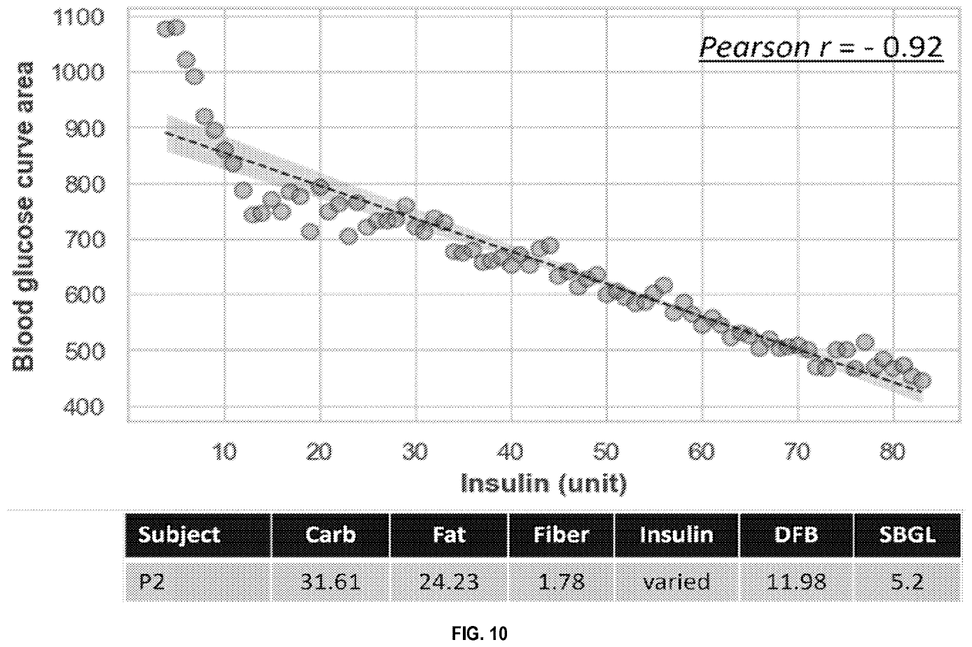

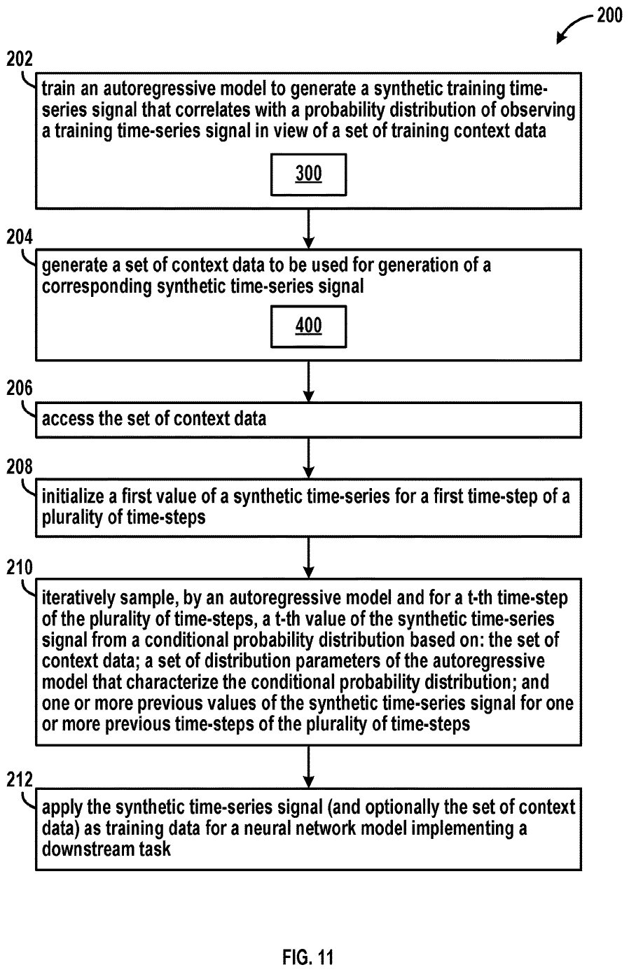

Referring to FIG. 2A, a system 100 implementing the framework for generating synthetic time-series physiological signal data can include a synthetic time-series generation component 110 that generates a synthetic time-series signal based on context data. The synthetic time-series generation component 110 can include an autoregressive model described in further detail herein. In the examples outlined herein, the synthetic time-series signal is a representation of a synthetic continuous glucose monitoring (CGM) signal. The synthetic time-series generation component 110 is operable for generating realistic synthetic time-series signals under the data scarcity conditions discussed in Section 2: Problem I above. The system 100 can further include a context generation component 120 that generates new context information (context data) which can be applied as input to the synthetic time-series generation component 110 to generate new synthetic time-series signals. The system 100 can be implemented at a computing device (e.g., computing device 500 shown in FIG. 10).

### A. Data Synthesis Model

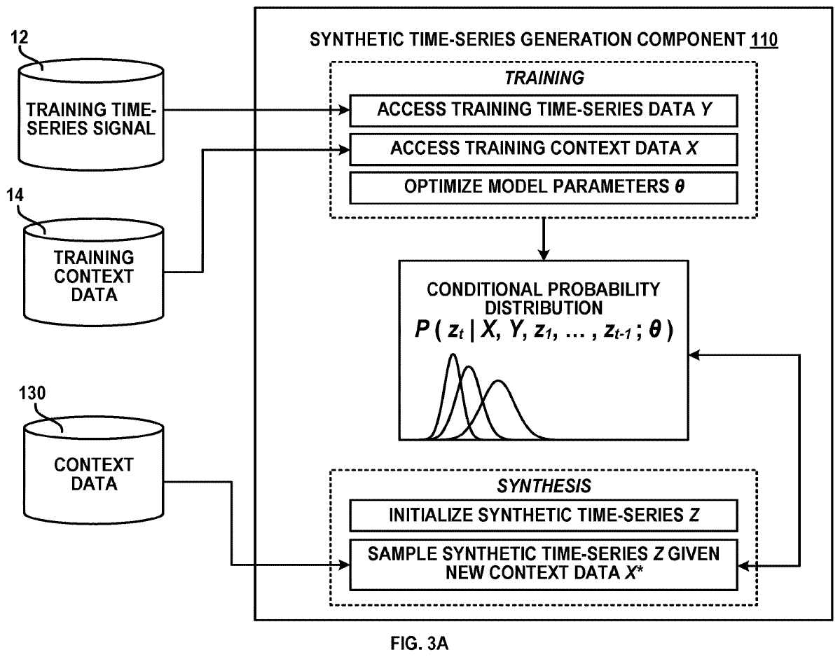

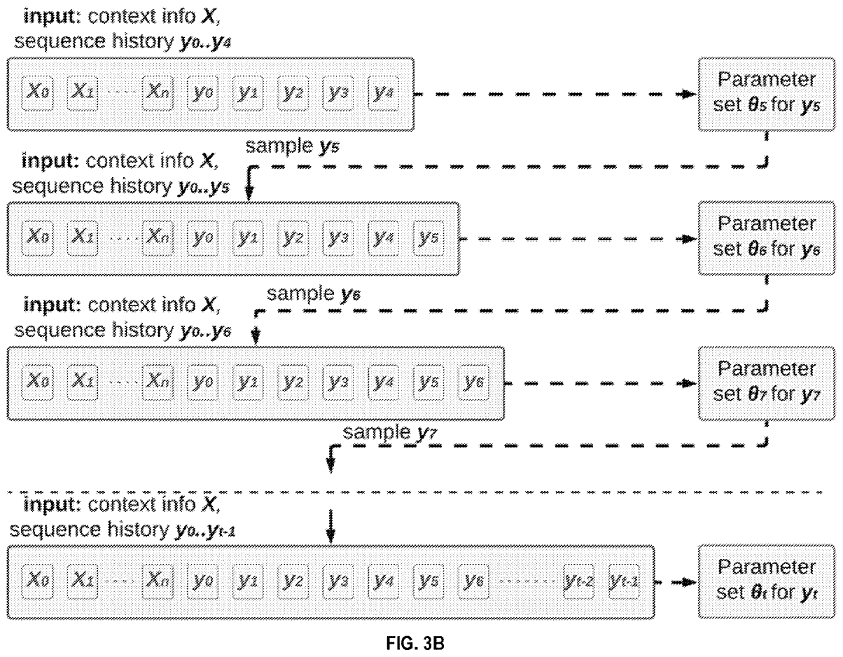

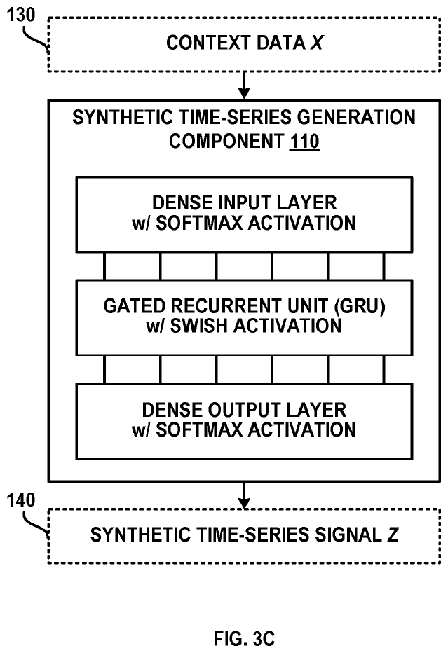

As shown in FIGS. 2A and 2B, and with additional reference to FIGS. 3A-3C, the synthetic time-series generation component 110 can access a training time-series signal 12 (y(t)∈Y) and a set of training context data 14 (given by X) from a training dataset 10, where the set of training context data 14 correlates with the training time-series signal 12. The training dataset 10 can be a relatively small dataset, as is the current state of available data outlined in the Problem Statement of Section 2 above. A training process of the synthetic time-series generation component 110 can include optimizing, over a plurality of training iterations, a set of distribution parameters θof the autoregressive model to generate a synthetic training time-series signal that correlates with a probability distribution of observing the training time-series signal 12 in view of the set of training context data 14. FIGS. 3A and 3B provide more information about the training process of the synthetic time-series generation component 110. FIG. 3C provides more information about the structure of the autoregressive model of the synthetic time-series generation component 110.

1) Approach: The autoregressive model learns the underlying patterns and relationships present in the data with conditional probability distribution P(yₜ|X, y₁, . . . , yₜ₋₁), where yₜ represents the t^{th} time-step in the real time-series sequence and X represents the set of contextual features. The autoregressive model uses the past time-steps y₁, y₂, . . . , yₜ₋₁ to predict the current time-step yₜ based on the contextual information in X. The objective is to learn the optimal parameters θ of the autoregressive model such that it can generate synthetic time-series sequences that consistently match the underlying probability distribution of the real data.

With reference to FIG. 3B, for any instance, the context X remains constant, but as the search for the parameters continues, the sequence history changes. If y₀, y₁, . . . , y₅ are present in the current history, the model determines the distribution parameter set θ₆ for item y₆. Sampling from the distribution produces y₆, and it becomes a part of the history. The procedure is then repeated for item y₇ and so on.

Once the learning phase is over, the model works on new contexts and synthesizes time-series Z. Mathematically, the autoregressive model can be formulated as:

P(Zₜ|X,Y,z₁, . . . ,zₜ₋₁);θ)=f(zₜ|g(X,Y,z₁, . . . ,zₜ₋₁);θ) (1)

where θ are the parameters of the autoregressive model, and where f and g are functions respectively associated with output and hidden layers of the model. The model is trained by maximizing the log-likelihood of the generated time-series Z with respect to the parameters θ.

$${arg max}_{θ} {\sum }_{i = 1}^{n} log P ( {z}_{t}^{i} \vert {X}^{i} , {Y}^{i} , {z}_{1}^{i} , … , {z}_{t - 1}^{i} ) ; θ ) \qquad ( 2 )$$

2) Neural Network Training: The autoregressive model can be implemented using neural networks, where the model parameters for all timestamps are learned during training. After initialization, the neural network parameters are iteratively optimized using all available training instances.

At any timestamp t, each sequence can be characterized by two parameters θ_{μ} and θ_{σ}. The two parameters respectively represent the mean and standard deviation of a Gaussian distribution from which the numerical value would be sampled. Optimizing the following loss function would make sure the synthesized value is close to the real-world value yₜ:

L(yₜ;μ,θ)=−log(f_{θ}_{_{μ}}_{θ}_{_{τ}}(yₜ)) (3)

FIG. 3C shows an example implementation of the autoregressive model of the synthetic time-series generation component 110, which can include a dense input layer that receives context data 130, followed by a gated recurrent unit (GRU), followed by a dense output layer that outputs a synthetic time series signal 140. The dense input and output layers can each incorporate a softmax activation function, and the GRU can incorporate a Swish activation function.

Algorithm 1 illustrates a training procedure for a single sequence dataset.
Algorithm 1: One training iteration at a single timestamp t
   Inputs:
   f←Neural Network
   X←context (Y)
   L←Loss
   for each timestamp yₜ do
      θ_{(t,0)}, θ_{(t,1)}, . . . ←f(X; y₀, y₁, . . . , yₜ₋₁)
      L←L+L(yₜ; θ₁, θ₂) . . . )
   end for

Each training iteration can include, at a t-th training iteration of the plurality of training iterations and for a t-th training time-step of the plurality of training time-steps:
a. determining a t-th set of distribution parameters θₜ of the set of distribution parameters θ of the autoregressive model based on the training time-series signal 12 (y(t));
b. sampling a t-th value zₜ of the synthetic training time-series signal (z(t)) from the t-th conditional probability distribution based on the set of training context data 14 and the t-th set of distribution parameters et; and
c. optimizing a loss function that compares the synthetic time-series signal (z(t)) with the training time-series signal (y(t) and maximizes a log-likelihood of observing the t-th value yₜ of the training time-series signal 12 (y(t)) under the t-th set of distribution parameters θₜ.

The t-th set of distribution parameters et characterize a t-th conditional probability distribution (see Eq. (1)) associated with observing a t-th value yₜ of the training time-series signal 12 (y(t)) under the set of training context data 14 and in view of one or more previous values (e.g., yₜ₋₁, yₜ₋₂, . . . , y₁) of the training time-series signal 12 for one or more previous training time-steps of the plurality of training time-steps. In other words, for each time step, the training process includes determining the t-th set of distribution parameters θₜ that would result in a sampled value zₜ that is close to yₜ. Then, once the t-th set of distribution parameters θₜ are determined for the time step, the training process includes sampling zₜ under the parameters θₜ to make sure that the parameters result in a value that is close to yₜ.

Following training, the synthetic time-series generation component 110 can access a set of new context data as input (e.g., context data 130, which can include new context data X* produced by the context generation component 120) and initialize a first value z₁ of a synthetic time-series signal 140 (z(t)) for a first time-step of a plurality of time-steps. The synthetic time-series generation component 110 can iteratively sample, by the autoregressive model and for a t-th time-step of a plurality of time-steps, a t-th value zₜ of a synthetic time-series signal 140 (z(t)) from a conditional probability distribution (Eq. (1) based on:
a. the set of context data 130 (which can include new context data X*);
b. the set of distribution parameters θₜ (including the t-th set of distribution parameters θₜ pertaining to the t-th time step) that characterize the conditional probability distribution; and
c. one or more previous values (e.g., zₜ₋₁, zₜ₋₂, . . . , z₁) of the synthetic time-series signal 140 for one or more previous time-steps.

### B. Generating Realistic Contextual Information

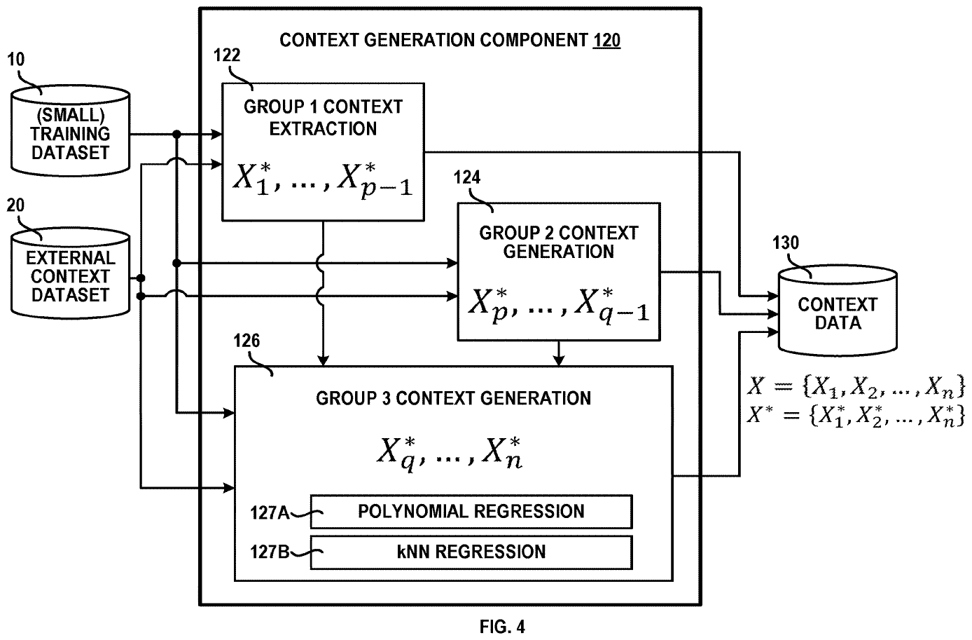

Referring to FIGS. 2A, 2B, and 4, the context generation component 120 can provide the synthetic time-series generation component 110 with (new) context data 130 in order to generate a synthetic time-series signal 140. As shown, the context generation component 120 can build from existing context data from an external context dataset 20 (and in some examples, context data present within the training dataset 10). The context data 130 generated by the context generation component 120 can include new context data denoted by

$${X}^{*} = \{ {X}_{1}^{*} , {X}_{2}^{*} , … , {X}_{n}^{*} \}$$

which will correlate with a synthetic time-series signal 140. The context data 130 for a synthetic time-series signal 140 can be divided up into groups based on type and availability of data.

As such, the context generation component 120 can include a “group 1 context extraction” component 122 that extracts a first group of context data X₁^{*}, . . . , Xₚ₋₁^{*} from an external source, e.g., the external context dataset 20 and/or the training dataset 10.

The context generation component 120 can further include a “group 2 context generation” component 124 that generates a second group of context data

$${X}_{p}^{*} , … , {X}_{q - 1}^{*}$$

within acceptable ranges that may be specific to each instance of context data within the second group. Context data within the second group would include data that does not necessarily depend on other types of context data and can therefore be randomly assigned within an acceptable range.

The context generation component 120 can further include a “group 3 context generation” component 126 that generates a third group of context data

$${X}_{q}^{*} , … , {X}_{n}^{*}$$

based on context data within the first group and/or the second group. The “group 3 context generation” component 126 can include machine learning elements that are operable for generating or otherwise inferring values of the third group of context data that would correlate with the context data within the first group and/or the second group. These machine learning elements can include a polynomial regression model 127A and/or a k-nearest neighbor (kNN) regression model 127B. Generation of each instance of context data within the third group, including which machine learning element would generate each instance, would depend on how the particular instance of context data would be expected to relate to other instances of context data as understood by experts.

1) Approach: With the autoregressive model of the synthetic time-series generation component 110 being trained on the labeled data to replicate its underlying representation, there is still a need for a large quantity of fresh contextual information to exploit this model and ensure volume production of synthetic data.

In the realm of digital healthcare, the context data can encompass a variety of information, such as sensor observations, medicinal information, genetic traits, social factors, environmental influences, demographics, and behavioral specifications. Some of these values may be randomly assigned within a certain range (e.g., the second group of context data), others require genuine data (e.g., the first group of context data), while the remainder (e.g., the third group of context data) can be predicted through basic machine learning techniques based on the available context. For example, while predicting a patient's risk of developing diabetes, age and gender might be readily available and can be randomly assigned within a certain range for a simulated patient (e.g., and can thus be included in the second group of context data generated by the “group 2 context generation” component 124). Others, like sensor observations and medication information, can be obtained from real data sources or medical records (e.g., and can thus be included in the first group of context data extracted by the “group 1 context extraction” component 122). However, there may be certain context values that are not directly available but can be predicted using basic machine learning techniques. For instance, the patient's physical activity history can be estimated based on accelerometer readings, and their dietary preferences can be identified through analysis of food consumption patterns. These types of context data can be included in the third group of context data generated by the “group 3 context generation” component 126)

In this case, real data from existing datasets is used as much as possible, then random values within range where applicable. The real data and the random values can then be applied to a polynomial regression or k-nearest neighbor (kNN) regression model to rough estimate the others. To be precise, the aim is to fill up the first group of context data

$${X}_{1}^{*} , … , {X}_{p - 1}^{*}$$

using real data from a different dataset, the second group of context data

$${X}_{p}^{*} , … , {X}_{q - 1}^{*}$$

using random values within range and use

$${X}_{1}^{*} , … , {X}_{q - 1}^{*}$$

as input to the polynomial regression model 127A or the kNN regression model 127B to estimate the rest, i.e., the third group of context data

$${X}_{q}^{*} , … , {X}_{n}^{*} .$$

The goal of polynomial regression is to find a polynomial function that best fits the data. When fitting a polynomial function for X_{q} with features X₁, . . . , X_{q-1} in the train data, the objective is to identify the set of coefficients β={β₁, β₁₂, . . . , β_{q-1}} such that the sum of squared errors is minimized:

$${arg min}_{β} {\sum }_{i} {( {X}_{iq} - {X}_{iq}^{*} )}^{2} \qquad ( 4 )$$

where,

$${X}_{iq}^{*} = {β}_{0} + {β}_{1} {X}_{i 1}^{*} + {β}_{12} {X}_{i 1}^{* 2} + {β}_{2} {X}_{i 2}^{*} + … + {β}_{mr} {X}_{im}^{* r} \qquad ( 5 )$$

kNN regression seeks to estimate the target value

$${X}_{iq}^{*}$$

by taking the average of the target values of its k-nearest neighbors in the training dataset:

$${X}_{iq}^{*} = \frac{1}{k} {\sum }_{i = 1}^{k} {X}_{iq} \qquad ( 6 )$$

The k-nearest neighbors are defined as the k training instances nearest to

$${X}_{i}^{*}$$

in terms of Euclidean distance:

$$arg {min}_{j = 1}^{k} {‖ {X}_{j} = {X}_{i}^{*} ‖}_{2} \qquad ( 7 )$$

In kNN regression, k is selected to be an odd number to avoid ties. The value of k can be chosen using cross-validation or other hyperparameter tuning techniques.

It is not always appropriate to assign random values within a range for all scenarios where one feature is correlated with others. This requires a clear understanding of the domain. For instance, it is highly unlikely that a person would take a high insulin dose after an intense workout session, based on professional knowledge of the subject matter. Thus, putting in a random value would thwart efforts to get quality data.

### 3. Implementation

In an example implementation of the system 100, two types of datasets were used. A first type of dataset is used to train the autoregressive model. A second type of dataset includes realistic contextual data that are fed to the autoregressive model to generate synthetic glucose signals.

1) Model training: The autoregressive model of the synthetic time-series generation component 110 was trained using Nutrition Absorption dataset, which includes CGM recordings from 5 participants (4 male, 1 female, Age: 51.6±16.1 y). All individuals had diabetes (4 T2DM, 1 T1DM) and they were provided a total of 167 meals under fasting condition. The dataset contains macronutrient amount of the meals (carbohydrates, fat, and fiber), time elapsed since the last insulin dosage (DFB), pre-meal blood glucose levels (SBGL), and CGM data spanning four hours post-meal, captured using the Medtrum S7 EasySense CGM System.

2) Context generation: In quest of an appropriate dataset for context generation, different options were explored. The US Department of Agriculture (USDA) National Nutrient Database for Standard Reference was found to be the most suitable. This dataset provides information on 8790 food items along with their corresponding macro and micro nutrients. The intent was to use this dataset as the basis of the context. However, the dataset needed tailoring to the purposes of this research by filtering out food items that were specific to infants and outside of the range of training data. For example, Babyfood-turkey, rice & veg, todd was removed as it is specific to babies and Poppyseed oil was removed as it includes no carbohydrate and is thus outside the range of training data. Moving forward, 3615 items were kept finally for further analysis.

3) Model Architecture: As shown in FIG. 3C, the architecture used for the autoregressive model of the synthetic time-series generation component 110 includes a gated recurrent unit (GRU) placed in between two dense layers (input and output). While the dense layers were equipped with Softplus activation function, the GRU layer had a Swish activation in place. The softplus activation function can be characterized as:

softplus(x)=log(1+exp(x)) (8)

and the Swish activation function can be written as:

$$swish ( x ) = x \cdot sigmoid ( β x ) = \frac{x}{1 + exp ( β x )} \qquad ( 9 )$$

where, x is the input to the activation function and β is a learnable parameter. However, putting β=1 is a common practice among researchers.

The softplus activation function is a smooth, non-linear activation function and very similar to the ReLU but differentiable at 0, which leads to better convergence during training. The rationale behind using Softplus is that it produces non-negative outputs which is compliant with requirements as blood glucose level cannot be negative.

For context generation, real food and their corresponding macro nutrients (carbohydrate, fat and fiber amounts) were used from the USDA dataset as part of the first group of context data. The other features (insulin intake, DFB, pre-meal blood glucose level) are generated based on the food information.

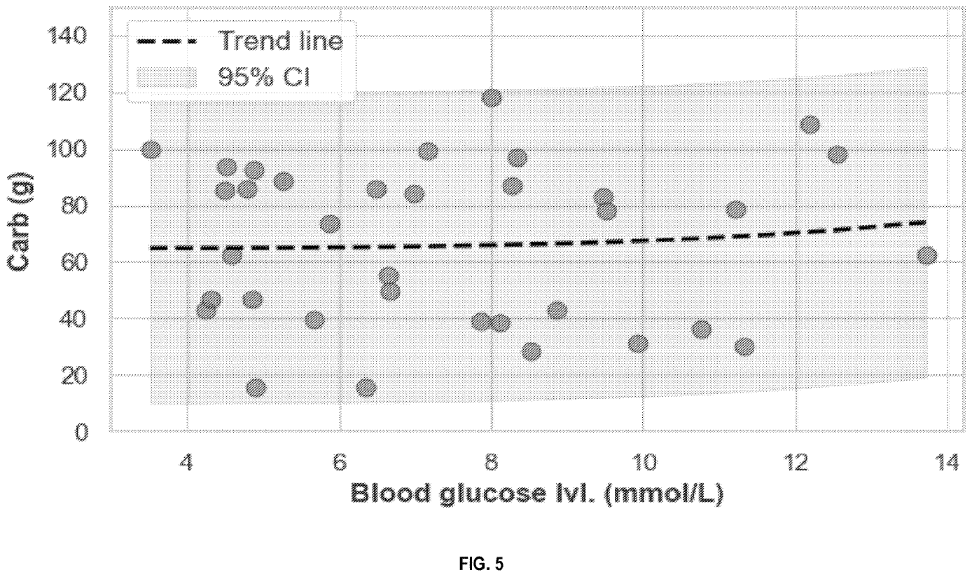

After analyzing the Nutrition Absorption dataset for each participant, no significant correlation was observed (P1: −0.05, P2: −0.29, P3: −0.08, P4: 0.02, P5: 0.02) between subjects' blood glucose levels and their carbohydrate (or fat/fiber) intake (FIG. 5). This means that the subjects' consumed foods were independent of their pre-meal blood glucose level. Therefore, it is reasonable to place random blood glucose levels under each corresponding context as part of the second group of context data.

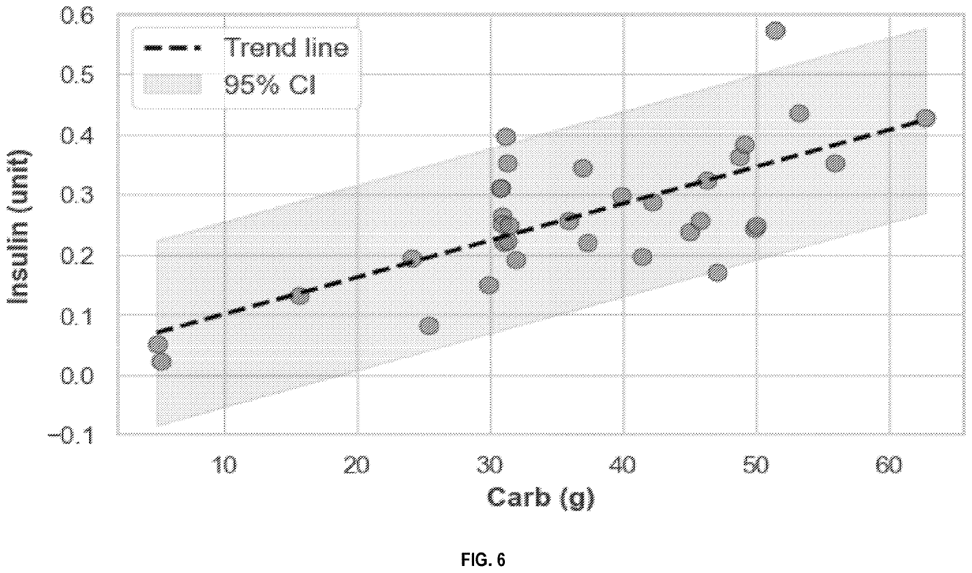

However, as shown in FIG. 6, this is not the case for insulin amounts taken right before the meals. Considerable positive correlation was observed between the insulin intakes and macro-nutrient amounts (P1: 0.3, P2: 0.76, P3: 0.4, P4: 0.71, P5: 0.21). Therefore, the polynomial regression model 127A was trained using macro-nutrients and insulin amounts from the Absorption dataset and then applied to impute insulin intakes for context generation based on the macro-nutrient amounts from the USDA. The information generated by the polynomial regression model 127A was included as part of the third group of context data.

Finally, no evidence was found that explains any existing relation between time since last insulin and other modalities. Thus, the preference was to use all other information (macro-nutrient amounts, insulin dose and pre-meal blood glucose level) in the kNN regression model 127B and produce rough estimates of time since last insulin to complete the contexts, for inclusion in the third group of context data.

A downstream machine learning model 150 (FIG. 2A) for validating the system 100 included a multi-task learning model for estimating the macro-nutrients. In one implementation, the multi-task learning model included a convolutional neural network (CNN) layer with 10 filters, a long short-term memory (LSTM) layer with 4 units and a dense leyer with 5 units before the output specific layers. The first two layers were equipped with rectified linear unit (ReLU) activation and adopted an Adam optimizer with an adaptive learning rate for the entire model. L2 regularizer and dropouts were used in the model to prevent overfitting. Thanks to data shortage, a 5-fold cross validation was performed for each subject. The reported results in the following section represent the average performance of the model across the 5-fold validation process. A clear separation between the test and train data was maintained both in autoregressive and multitask model training to ensure integrity and prevent data leakage during 5-fold validation. The metric used for evaluating the regression task is root mean square relative error (RMSRE):

$$RMSRE = \sqrt{\frac{1}{s} \sum \frac{{( y - \hat{y} )}^{2}}{{y}^{2}}} \qquad ( 10 )$$

where s is the number of samples, y refers to the true quantity and ŷ is the estimated one. Since the dataset has different quantities of carbohydrates, fat, and fiber, using RMSRE is reasonable.

### 4. Results of Validation Study

Results presented herein are on the basis of statistical measures, sensitivity analysis, and a downstream macro-nutrient estimator.

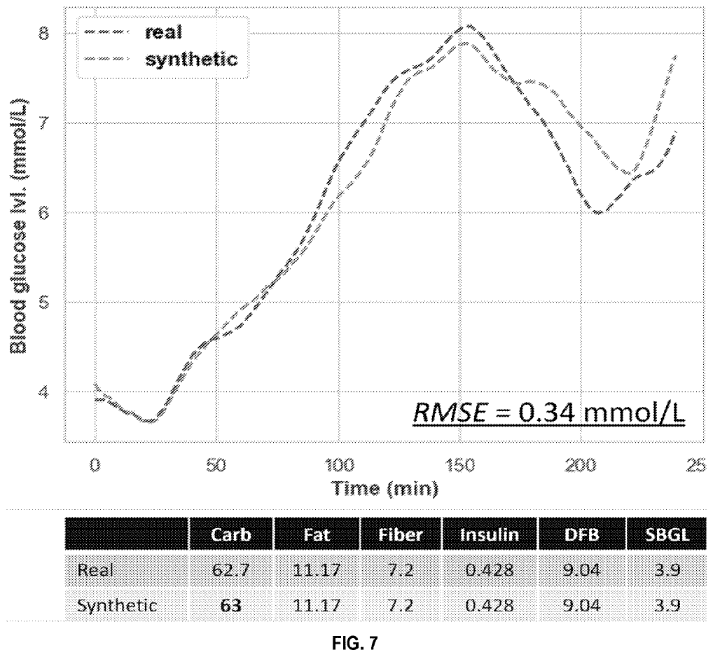

With the Nutrient Absorption dataset, synthetic CGMs were produced for each participant individually and each CGM signal corresponds to a context (set consisting of meal information, insulin information and blood glucose level) developed from USDA data. FIG. 7 shows a synthetic CGM generated by adding minuscule noise to the context of a real one.

### A. Statistical Performance

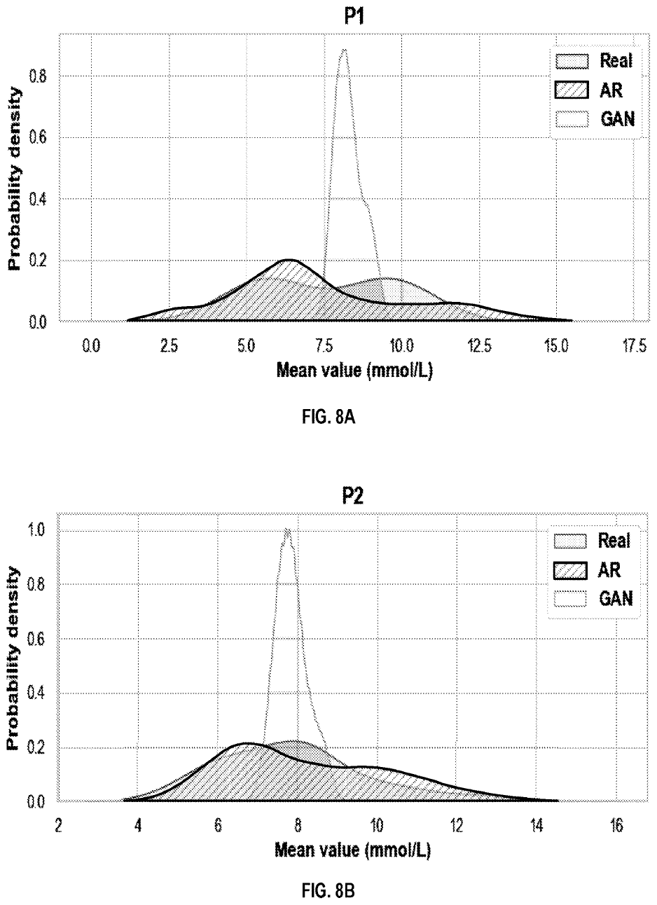

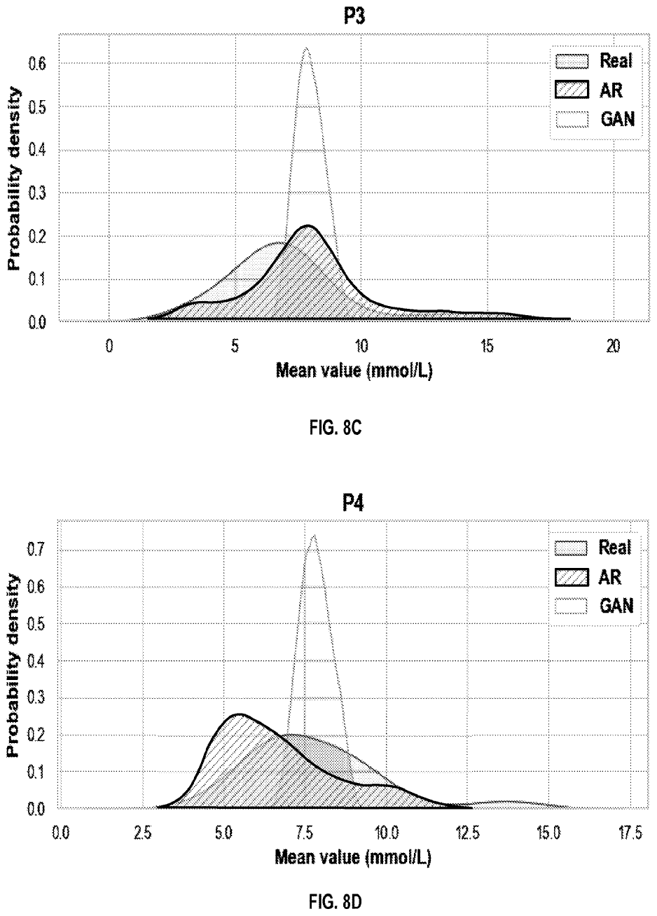

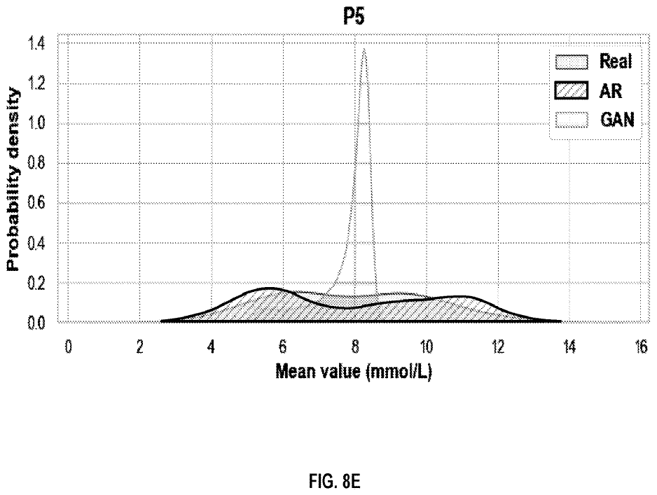

The quality of the synthetic CGM data generated using the autoregressive model is validated by comparing their statistical measures against those of the real data and synthetic data generated by GAN. Among these measures, the distribution of the mean values for each subject is visualized using Kernel Density Estimate (KDE) plots. As shown in FIGS. 8A-8E, the mean values of the synthetic data are comparable to those of the real data for all subjects. This close resemblance indicates that the AR method effectively captures the underlying data characteristics. In contrast, the GAN-generated data shows a noticeable deviation from the real data distribution and suggests GAN's less effectiveness in mimicking the true data patterns.

In addition to the mean values, other metrics like Time in range (TIR), % Time in hyper, % Time in L1 and L2 hypo and standard deviation of the signals were also used for comparison and depicted in Table I. The thresholds for hyper (BGL≥9.99 mmol/L), TIR (9.99 mmol/L>BGL≥3.9 mmol/L), L1 hypo (3.9 mmol/L>BGL≥3.0 mmol/L) and L2 hypo (3.0 mmol/L>BGL) are used to categorize the blood glucose levels into different bins. The aforementioned metrics provide valuable insights into the overall characteristics and accuracy of the synthetic data generated with the autoregressive model. As seen from FIGS. 8A-8E and Table I, synthetic data from the AR model is more skewed like the real data compared to the synthetic data from the GAN which is more centered towards the mean value and hardly has anything below the hypoglycemic threshold.

TABLE I: Comparison of different statistical parameters (Mean, TIR, Time in hyper, Time in L1 and L2 hypo, SD) stemming from Real and Synthetic CGM signals for five subjects.

TABLE I
Comparison of different statistical parameters (Mean, TIR,
Time in hyper, Time in L1 and L2 hypo, SD) stemming from
Real and Synthetic CGM signals for five subjects.

| | | Metric | | | | | |
|---|---|---|---|---|---|---|---|
| | | Mean | | | L1 | L2 | SD |
| | | (mmol/ | TIR | Hyper | Hypo | Hypo | (mmol/ |
| Subject | | L) | (%) | (%) | (%) | (%) | L) |
| P1 | Real | 7.62 | 70.83 | 21.78 | 5.37 | 2.01 | 0.66 |
| | AR synth. | 7.32 | 61.98 | 21.19 | 6.14 | 10.69 | 0.79 |
| | GAN synth. | 8.3 | 96.7 | 3.344 | 0.0 | 0.0 | 0.71 |
| P2 | Real | 7.86 | 81.31 | 17.08 | 1.59 | 0.02 | 0.65 |
| | AR synth. | 8.22 | 77.42 | 21.03 | 0.05 | 0.69 | 0.47 |
| | GAN synth. | 7.85 | 99.65 | 0.35 | 0 | 0 | 0.36 |
| P3 | Real | 7.26 | 77.41 | 13.95 | 5.22 | 3.43 | 0.75 |
| | AR synth. | 8.18 | 70.33 | 20.18 | 0.05 | 3.47 | 0.76 |
| | GAN synth. | 7.95 | 99.21 | 0.79 | 0 | 0 | 0.61 |
| P4 | Real | 7.80 | 81.54 | 16.94 | 0.98 | 0.54 | 0.41 |
| | AR synth. | 6.70 | 81.21 | 12.64 | 0.01 | 1.93 | 0.13 |
| | GAN synth. | 7.83 | 99.45 | 0.55 | 0 | 0 | 0.35 |
| P5 | Real | 7.91 | 66.07 | 27.5 | 6.14 | 0.29 | 0.62 |
| | AR synth. | 7.89 | 54.82 | 33.72 | 0.08 | 4.32 | 0.36 |
| | GAN synth. | 7.83 | 98.55 | 0.62 | 0 | 0.83 | 0.84 |

### B. Sensitivity Analysis

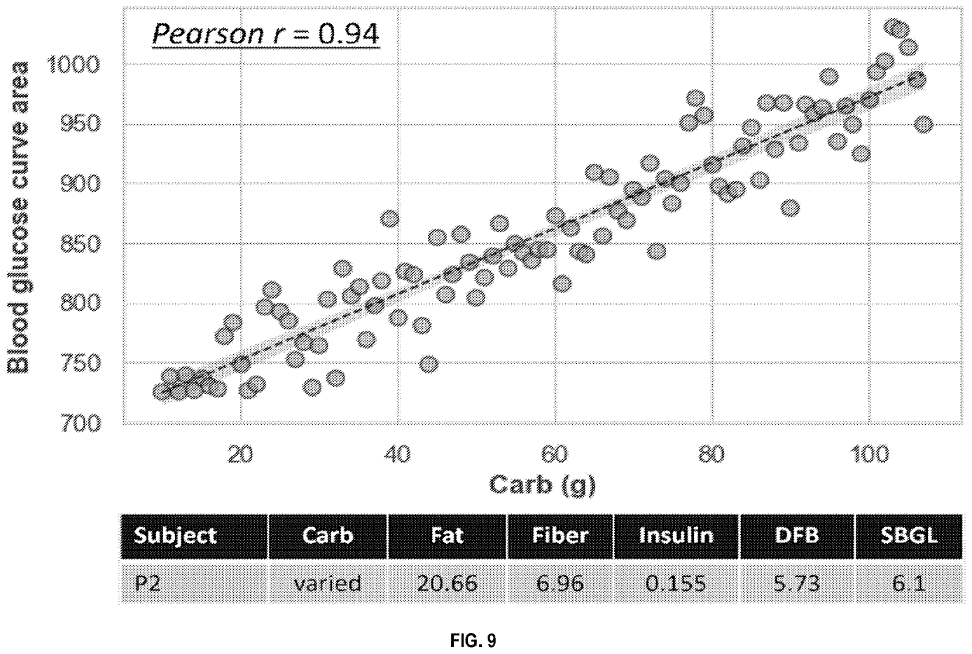

A sensitivity analysis is conducted to understand whether manipulating individual features in a specific direction leads to expected changes in the model's output. Real contexts are selected, their carb and insulin amounts are varied across the full range, fed to the model and the mean/area under the CGM curve (AUC)/peak of the output CGM are monitored. As documented in prior research, blood glucose level increases and decreases with increase in carb and insulin amounts, respectively. Findings from the sensitivity analysis are consistent with them. This test indicates that the trained autoregressive models outlined herein follow the expected norm. FIGS. 9 and 10 depict the scatter plots related to the sensitivity of one of the samples along with the associated correlation value. “Table II presents the overall results of the sensitivity analysis. The reported numbers represent the average correlation coefficients. To compute these averages, the carbohydrate and insulin intake was varied for all samples from the subjects, and these manipulated contexts were fed to the model to calculate the Pearson correlation coefficients for the mean, AUC, and peak values of the resulting CGM signals. The correlation coefficients are then transformed using the Fisher Z transformation to calculate their average, which is subsequently inversely transformed back to Pearson correlation coefficients.”

TABLE II
Results on sensitivity analysis performed by monitoring mean,
AUC and peak CGMs while varying the CHO and insulin intakes.

| | Manipulating CHO | | | Manipulating Insulin | | |
|---|---|---|---|---|---|---|
| Subject | mean | AUC | peak | mean | AUC | peak |
| P1 | 0.9 ± | 0.91 ± | 0.95 ± | −0.93 ± | −0.88 ± | −0.9 ± |
| | 0.08 | 0.07 | 0.12 | 0.04 | 0.06 | 0.16 |
| P2 | 0.87 ± | 0.91 ± | 0.88 ± | −0.86 ± | −0.84 ± | −0.87 ± |
| | 0.13 | 0.05 | 0.07 | 0.1 | 0.11 | 0.14 |
| P3 | 0.81 ± | 0.78 ± | 0.82 ± | −0.83 ± | −0.83 ± | −0.85 ± |
| | 0.24 | 0.17 | 0.16 | 0.13 | 0.11 | 0.14 |
| P4 | 0.94 ± | 0.91 ± | 0.9 ± | −0.86 ± | −0.9 ± | −0.87 ± |
| | 0.08 | 0.09 | 0.11 | 0.12 | 0.11 | 0.15 |
| P5 | 0.89 ± | 0.93 ± | 0.88 ± | −0.91 ± | −0.84 ± | −0.88 ± |
| | 0.13 | 0.1 | 0.14 | 0.09 | 0.16 | 0.12 |

### C. Downstream Task Performance

At this point, the synthetic CGM data was fitted in a multitask learning network to see how it performs in a downstream task compared to its real counterpart. Given the entire CGM signal, the task of the multi-task learning network is to estimate the meal macro nutrients (carb, fat and fiber). These downstream task evaluations aimed to provide evidence supporting the quality and usability of the synthetic CGM data in an application domain. Comparing the performance of synthetic blend with that of real CGM data sought to answer the extent to which the blend could serve as a reliable substitute. Referring to Table III, notably, there are instances where the synthetic data outperforms the real data based on RMSRE. P3 exhibits relatively poor RMSRE values for all three macro-nutrients because P3 had the least number of samples and the lowest HbA1c percentage compared to the rest of the participants. However, subjects P1, P2, P4, and P5 generally manifested better estimation accuracy, with lower RMSRE values for certain macro-nutrients. Error is substantially lower for carbohydrate estimation due to direct correlation between carbohydrate intake and CGM readings. Overall, the table highlights the potential of synthetic data in enhancing the accuracy of macro-nutrient estimations, with some exceptions observed across different subjects and macronutrients. In addition to comparable or superior estimation accuracy for most subjects, inclusion of the synthetic samples resulted in enhanced training stability and consistent estimations for unseen samples.

TABLE III: Performance evaluation of synthetic CGMs from different techniques against the real ones in a downstream macronutrient estimator model for all five subjects.

TABLE III
Performance evaluation of synthetic CGMs from different
techniques against the real ones in a downstream macronutrient
estimator model for all five subjects.

Macro-nutrient

| Subject | Data | carb (g) | fat (g) | fiber (g) |
|---|---|---|---|---|
| P1 | Real | 0.43 | 0.54 | 0.63 |
| | Real + AR synth. | 0.21 | 0.58 | 0.52 |
| | Real + GAN synth. | 0.95 | 1.06 | 1.22 |
| P2 | Real | 0.43 | 0.79 | 0.92 |
| | Real + AR synth. | 0.42 | 0.68 | 0.21 |
| | Real + GAN synth. | 0.86 | 1.13 | 0.92 |
| P3 | Real | 0.64 | 1.98 | 1.09 |
| | Real + AR synth. | 0.61 | 1.98 | 1.05 |
| | Real + GAN synth. | 1.17 | 2.31 | 1.47 |
| P4 | Real | 0.57 | 0.65 | 0.44 |
| | Real + AR synth. | 0.60 | 0.61 | 0.32 |
| | Real + GAN synth. | 0.74 | 0.83 | 1.15 |
| P5 | Real | 0.52 | 0.71 | 0.6 |
| | Real + AR synth. | 0.43 | 0.7 | 0.68 |
| | Real + GAN synth. | 0.72 | 1.88 | 1.27 |

### 4. Computer-implemented System and Methods

### Methods

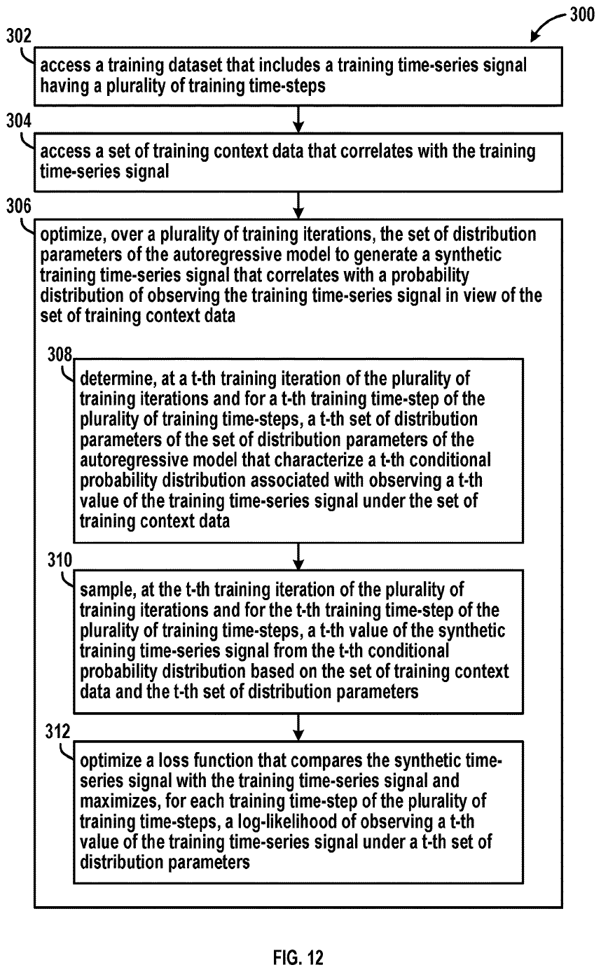

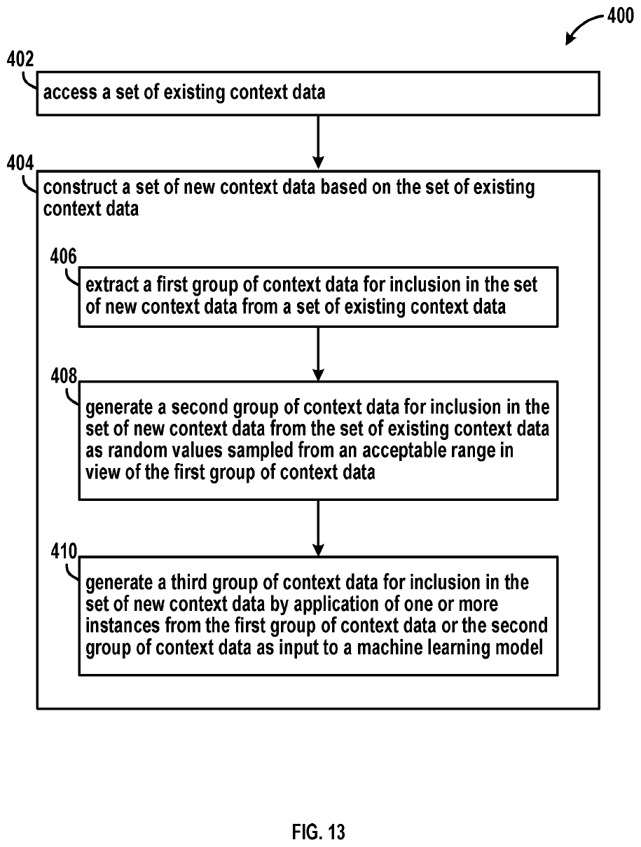

FIGS. 11-13 are a series of process flow diagrams illustrating a method 200 for generating synthetic time-series physiological signal data by the system 100 of FIGS. 2A-4. Method 200 of FIG. 11 includes a sub-method 300 expanded on in FIG. 12, where sub-method 300 is directed to training the autoregressive model of the synthetic time-series generation component 110. Further, method 200 of FIG. 11 includes a sub-method 400 expanded on in FIG. 13, where sub-method 400 is directed to generating new context data for application as input to the synthetic time-series generation component 110.

Referring to FIG. 11, step 202 of method 200 includes training an autoregressive model to generate a synthetic training time-series signal that correlates with a probability distribution of observing a training time-series signal in view of a set of training context data. Step 202 includes various sub-steps discussed further herein as sub-method 300 of FIG. 12. Step 204 of method 200 includes generating the set of context data to be used for generation of a corresponding synthetic time-series signal. Step 204 likewise includes various sub-steps discussed further herein as sub-method 400 of FIG. 13.

Step 206 of method 200 includes accessing the set of context data (e.g., by the synthetic time-series generation component 110). Step 208 of method 200 includes initializing a first value of a synthetic time-series for a first time-step of a plurality of time-steps.

Step 210 of method 200 includes iteratively sampling, by the autoregressive model and for a t-th time-step of the plurality of time-steps, a t-th value of the synthetic time-series signal from a conditional probability distribution based on: the set of context data; a set of distribution parameters of the autoregressive model that characterize the conditional probability distribution; and one or more previous values of the synthetic time-series signal for one or more previous time-steps of the plurality of time-steps.

Step 212 of method 200 includes applying the synthetic time-series signal (and optionally the set of context data) as training data for a neural network model implementing a downstream task.

Referring to FIG. 12, and with additional reference to FIG. 11, the sub-method 300 implements aspects of step 202 of FIG. 11 directed to training the autoregressive model. Step 302 of sub-method 300 includes accessing a training dataset that includes a training time-series signal having a plurality of training time-steps. Step 304 of sub-method 300 includes accessing a set of training context data that correlates with the training time-series signal. Step 306 of sub-method 300 includes optimizing, over a plurality of training iterations, the set of distribution parameters of the autoregressive model to generate a synthetic training time-series signal that correlates with a probability distribution of observing the training time-series signal in view of the set of training context data.

Step 306 of sub-method 300 includes various sub-steps that are iteratively repeated at each training iteration, including steps 308-312.

Step 308 includes determining, at a t-th training iteration of the plurality of training iterations and for a t-th training time-step of the plurality of training time-steps, a t-th set of distribution parameters of the set of distribution parameters of the autoregressive model, the t-th set of distribution parameters characterizing a t-th conditional probability distribution associated with observing a t-th value of the training time-series signal under the set of training context data and in view of one or more previous values of the training time-series signal for one or more previous training time-steps of the plurality of training time-steps.

Step 310 includes sampling, at the t-th training iteration of the plurality of training iterations and for the t-th training time-step of the plurality of training time-steps, a t-th value of the synthetic training time-series signal from the t-th conditional probability distribution based on the set of training context data and the t-th set of distribution parameters.

Step 312 includes optimizing a loss function that compares the synthetic time-series signal with the training time-series signal and maximizes, for each training time-step of the plurality of training time-steps, a log-likelihood of observing a t-th value of the training time-series signal under a t-th set of distribution parameters.

Referring to FIG. 13, and with additional reference to FIG. 11, the sub-method 400 implements aspects of step 204 of FIG. 11 directed to context data generation. Step 402 of sub-method 400 includes accessing a set of existing context data. Step 404 of sub-method 400 includes constructing a set of new context data based on the set of training context data. Step 404 of sub-method 400 includes various sub-steps, including steps 406-410.

Step 406 includes extracting a first group of context data for inclusion in the set of new context data from a set of existing context data.

Step 408 includes generating a second group of context data for inclusion in the set of new context data from the set of existing context data as random values sampled from an acceptable range in view of the first group of context data.

Step 410 includes generating a third group of context data for inclusion in the set of new context data by application of one or more instances from the first group of context data or the second group of context data as input to a machine learning model.

### Computing Device

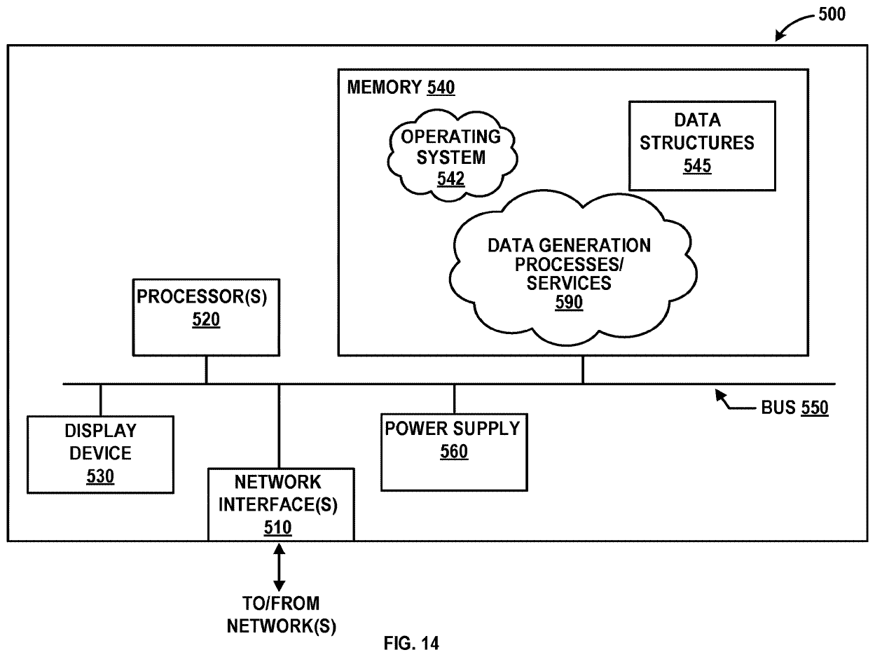

FIG. 14 is a schematic block diagram of an example computing device 500 that may be used with one or more embodiments described herein, e.g., as a component of system 100 shown in FIGS. 2A and 2B.

Computing device 500 comprises one or more network interfaces 510 (e.g., wired, wireless, PLC, etc.), at least one processor 520, and a memory 540 interconnected by a system bus 550, as well as a power supply 560 (e.g., battery, plug-in, etc.). Computing device 500 can also include or otherwise communicate with a display device 530 for displaying a graphical user interface and/or representations of the results of the methods outlined herein.

Network interface(s) 510 include the mechanical, electrical, and signaling circuitry for communicating data over the communication links coupled to a communication network. Network interfaces 510 are configured to transmit and/or receive data using a variety of different communication protocols. As illustrated, the box representing network interfaces 510 is shown for simplicity, and it is appreciated that such interfaces may represent different types of network connections such as wireless and wired (physical) connections. Network interfaces 510 are shown separately from power supply 560, however it is appreciated that the interfaces that support PLC protocols may communicate through power supply 560 and/or may be an integral component coupled to power supply 560.

Memory 540 includes a plurality of storage locations that are addressable by processor 520 and network interfaces 510 for storing software programs and data structures associated with the embodiments described herein. In some embodiments, device 500 may have limited memory or no memory (e.g., no memory for storage other than for programs/processes operating on the device and associated caches). Memory 540 can include instructions executable by the processor 520 that, when executed by the processor 520, cause the processor 520 to implement aspects of the system 100 and methods (e.g., method 200, sub-method 300 and sub-method 400) outlined herein.

Processor 520 comprises hardware elements or logic adapted to execute the software programs (e.g., instructions) and manipulate data structures 545. An operating system 542, portions of which are typically resident in memory 540 and executed by the processor, functionally organizes device 500 by, inter alia, invoking operations in support of software processes and/or services executing on the device. These software processes and/or services may include data generation processes/services 590, which can include aspects of method 200, sub-method 300 and sub-method 400 and/or implementations of various modules including the synthetic time-series generation component 110 and/or the context generation component 120 of the system 100 described herein. Note that while data generation processes/services 590 is illustrated in centralized memory 540, alternative embodiments provide for the process to be operated within the network interfaces 510, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the data generation processes/services 590 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method for generating synthetic time-series physiological signal data, comprising:
accessing a set of context data; and
iteratively sampling, by an autoregressive model and for a t-th time-step of a plurality of time-steps, a t-th value of a synthetic time-series signal from a conditional probability distribution based on:
the set of context data;
a set of distribution parameters of the autoregressive model that characterize the conditional probability distribution; and
one or more previous values of the synthetic time-series signal for one or more previous time-steps of the plurality of time-steps.

2. The method of claim 1, further comprising:
applying the synthetic time-series signal as training data for a neural network model implementing a downstream task.

3. The method of claim 1, the synthetic time-series signal being a representation of a synthetic continuous glucose monitoring (CGM) signal.

4. The method of claim 1, further comprising:
accessing a training dataset that includes a training time-series signal having a plurality of training time-steps;
accessing a set of training context data that correlates with the training time-series signal; and
optimizing, over a plurality of training iterations, the set of distribution parameters of the autoregressive model to generate a synthetic training time-series signal that correlates with a probability distribution of observing the training time-series signal in view of the set of training context data.

5. The method of claim 4, further comprising:
determining, at a t-th training iteration of the plurality of training iterations and for a t-th training time-step of the plurality of training time-steps, a t-th set of distribution parameters of the set of distribution parameters of the autoregressive model, the t-th set of distribution parameters characterizing a t-th conditional probability distribution associated with observing a t-th value of the training time-series signal under the set of training context data and in view of one or more previous values of the training time-series signal for one or more previous training time-steps of the plurality of training time-steps; and
sampling, at the t-th training iteration of the plurality of training iterations and for the t-th training time-step of the plurality of training time-steps, a t-th value of the synthetic training time-series signal from the t-th conditional probability distribution based on the set of training context data and the t-th set of distribution parameters.

6. The method of claim 4, further comprising:
optimizing a loss function that compares the synthetic training time-series signal with the training time-series signal and maximizes, for each training time-step of the plurality of training time-steps, a log-likelihood of observing a t-th value of the training time-series signal under a t-th set of distribution parameters.

7. The method of claim 1, further comprising:
accessing a set of existing context data; and
constructing a set of new context data based on the set of existing context data.

8. The method of claim 7, the set of context data for sampling the t-th value of the synthetic time-series signal including the set of new context data.

9. The method of claim 7, further comprising:
generating a third group of context data for inclusion in the set of new context data by application of one or more instances from a first group of context data or a second group of context data as input to a machine learning model.

10. The method of claim 9, further comprising:
extracting the first group of context data for inclusion in the set of new context data from the set of existing context data.

11. The method of claim 9, further comprising:
generating the second group of context data for inclusion in the set of new context data from the set of existing context data as random values sampled from an acceptable range in view of the first group of context data.

12. The method of claim 9, the machine learning model including a polynomial regression model.

13. The method of claim 9, the machine learning model including a kNN regression model.

14. The method of claim 9, the third group of context data including insulin dose amount generated in view of the first group of context data including food items and corresponding macronutrients associated with the food items.

15. The method of claim 9, the third group of context data including time since last insulin dose generated in view of one or more of:
the first group of context data including food items and corresponding macronutrients associated with the food items;
the second group of context data including pre-meal blood glucose levels; and
the third group of context data including insulin dose amount.

16. A system for generating synthetic time-series physiological signal data, comprising:
a processor in communication with a memory, the memory including instructions executable by the processor to:
access a set of context data; and
iteratively sample, by an autoregressive model and for a t-th time-step of a plurality of time-steps, a t-th value of a synthetic time-series signal from a conditional probability distribution based on:
the set of context data;
a set of distribution parameters of the autoregressive model that characterize the conditional probability distribution; and
one or more previous values of the synthetic time-series signal for one or more previous time-steps of the plurality of time-steps.

17. The system of claim 16, the synthetic time-series signal being a representation of a synthetic continuous glucose monitoring (CGM) signal.

18. The system of claim 16, the memory further including instructions executable by the processor to:
access a training dataset that includes a training time-series signal having a plurality of training time-steps;
access a set of training context data that correlates with the training time-series signal; and
optimize, over a plurality of training iterations, the set of distribution parameters of the autoregressive model to generate a synthetic training time-series signal that correlates with a probability distribution of observing the training time-series signal in view of the set of training context data.

19. The system of claim 18, the memory further including instructions executable by the processor to:
determine, at a t-th training iteration of the plurality of training iterations and for a t-th training time-step of the plurality of training time-steps, a t-th set of distribution parameters of the set of distribution parameters of the autoregressive model, the t-th set of distribution parameters characterizing a t-th conditional probability distribution associated with observing a t-th value of the training time-series signal under the set of training context data and in view of one or more previous values of the training time-series signal for one or more previous training time-steps of the plurality of training time-steps;
sample, at the t-th training iteration of the plurality of training iterations and for the t-th training time-step of the plurality of training time-steps, a t-th value of the synthetic training time-series signal from the t-th conditional probability distribution based on the set of training context data and the t-th set of distribution parameters; and
optimize a loss function that compares the synthetic time-series signal with the training time-series signal and maximizes, for each training time-step of the plurality of training time-steps, a log-likelihood of observing the t-th value of the training time-series signal under the t-th set of distribution parameters.

20. The system of claim 16, the memory further including instructions executable by the processor to:
access a set of existing context data; and
construct a set of new context data for inclusion within the set of context data for sampling the synthetic time-series signal based on the set of existing context data, the set of new context data including a third group of context data generated by application of one or more instances from a first group of context data or a second group of context data as input to a machine learning model.
